(19) United States
(12) Patent Application Publication
(10) Pub. No.: US 2009/0175792 A1
(43) Pub. Date: Jul. 9, 2009

(54) GLYCOMIMETIC INHIBITORS OF SIGLEC-8

(75) Inventors: John L. Magnani, Gaithersburg, MD (US); Arun K. Sarkar, North Potomac, MD (US)

Correspondence Address: SEED INTELLECTUAL PROPERTY LAW GROUP PLLC, 701 FIFTH AVE, SUITE 5400, SEATTLE, WA, 98104, US

(73) Assignee: GlycoMimetics, Inc., Gaithersburg, MD (US)

(21) Appl. No.: 12/304,879

(22) PCT Filed: Jun. 19, 2007
(86) PCT No.: PCT/US07/14457

§ 371 Date: Dec. 15, 2008

Related U.S. Application Data
(60) Provisional application No. 60/816,203, filed on Jun. 23, 2006.
(60) Provisional application No. 60/925,500, filed on Apr. 20, 2007.

(51) Int. Cl. A61K 49/00; C12N 5/02; A61K 31/35

(52) U.S. Cl. 424/9.1; 435/375; 514/459

(57) ABSTRACT

Compounds, compositions and methods are provided for detecting or modulating in vitro and in vivo processes mediated by Siglec-8 binding. More specifically, Siglec-8 modulators and their use are described, wherein the Siglec-8 modulators that modulate a Siglec-8-mediated function comprise particular glycomimetics alone or linked to a diagnostic or therapeutic agent.

### BACKGROUND

1. Technical Field

The present invention relates generally to compounds, compositions and methods for modulating or detecting processes mediated by Siglec-8 binding, and more particularly to Siglec-8 modulators and their use, wherein the Siglec-8 modulators that modulate a Siglec-8-mediated function comprise particular glycomimetics alone or linked to a diagnostic or therapeutic agent.

2. Description of the Related Art

Siglec-8 is a mammalian lectin from a family of 1-type lectins that consist of similar sequence homology (Siglecs). Siglec-8 has been reported by the Glycomics Consortium to bind 6′ sulfated sialyl Le^{x}. As 6′ sulfated sialyl Le^{x} contains fucose, it is believed that fucose is necessary for its binding Siglec-8.

Siglec-8 is a specific marker for human eosinophils, basophils and mast cells. These cells are known to be involved in a number of diseases, particularly inflammatory diseases. Inflammatory diseases include asthma, psoriasis and allergy. Inhibitors of Siglec-8 may regulate the functions of Siglec-8-expressing cells, including effects on their survival. Therefore, inhibitors of Siglec-8 have therapeutic potential for diseases such as asthma.

Accordingly, there is a need in the art for identifying inhibitors of Siglec-8 and for the development of methods employing such compounds to inhibit the development of conditions associated with Siglec-8. The present invention fulfills these needs and further provides other related advantages.

### BRIEF SUMMARY

Briefly stated, this invention provides compounds, compositions and methods for modulating or detecting processes mediated by Siglec-8. In the present invention, the compounds that modulate (e.g., inhibit or enhance) or detect a Siglec-8-mediated function comprise, or consist of, a particular glycomimetic alone or linked to a diagnostic or therapeutic agent. Such compounds may be combined with a pharmaceutically acceptable carrier or diluent to form a pharmaceutical composition. The compounds or compositions may be used in a method to modulate or detect a Siglec-8-mediated function.

In one embodiment of the present invention, a compound for use in the methods is with the formula:

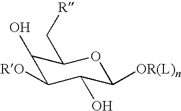

wherein
R═N-acetylglucoseamine or

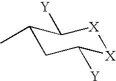

wherein both X are CHY, or one X is O and the other X is CHY, where the Y are independently selected from H, OH, NHZ, (CH₂)_{q}OSO₃, and CH₂Z, where Z is selected from or are independently selected from H, acetyl, aliphatic group and aromatic group;
q=0-4;
L=linker group;
n=0-1;

R′═H, sialic acid, or a sialic acid analog;
R″═OH, sulfated-(A)ₘ group, carboxylated-(A)ₘ group, or phosphorylated-(A)ₘ group;
A=aliphatic or aromatic group; and
m=0-1.

In another embodiment, the above compound is in combination with a pharmaceutically acceptable carrier or diluent.

In another embodiment, the above compound is linked by L (n=1) to a diagnostic agent.

In another embodiment, the above compound is linked by L (n=1) to a therapeutic agent.

In other embodiments, particular methods for using the above compound or compositions thereof are provided. In certain embodiments, the methods of use may comprise, or consist of, the following methods. The compound may be used in a method for modulating the activity of Siglec-8 comprising, or consisting of, contacting a cell with an effective amount of the compound. The compound may be used in a method of treating a patient who is in need of having inhibited the development of a condition associated with Siglec-8, comprising, or consisting of, administering to the patient the compound in an amount effective to inhibit the development of such a condition. The compound may be used in a method of targeting an agent to a cell expressing Siglec-8, comprising, or consisting of, contacting the cell with the compound in an amount effective to target a diagnostic or therapeutic agent to the cell, wherein the agent is linked by L (n=1) to the compound. The compound may be used in a method of targeting an agent to a cell expressing Siglec-8 in a patient in need thereof, comprising, or consisting of, administering to the patient a compound in an amount effective to target a diagnostic or therapeutic agent to the cell, wherein the agent is linked by L (n=1) to the compound. In any of the embodiments of the present invention, the compound may be in combination with a pharmaceutically acceptable carrier or diluent.

In other embodiments, the above compounds or compositions thereof may be used in the manufacture of a medicament, for example for any of the uses recited above.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 is a diagram illustrating the synthesis of an embodiment of Siglec-8 modulating compound.

FIG. 2 is a diagram illustrating the synthesis of an embodiment of Siglec-8 modulating compound.

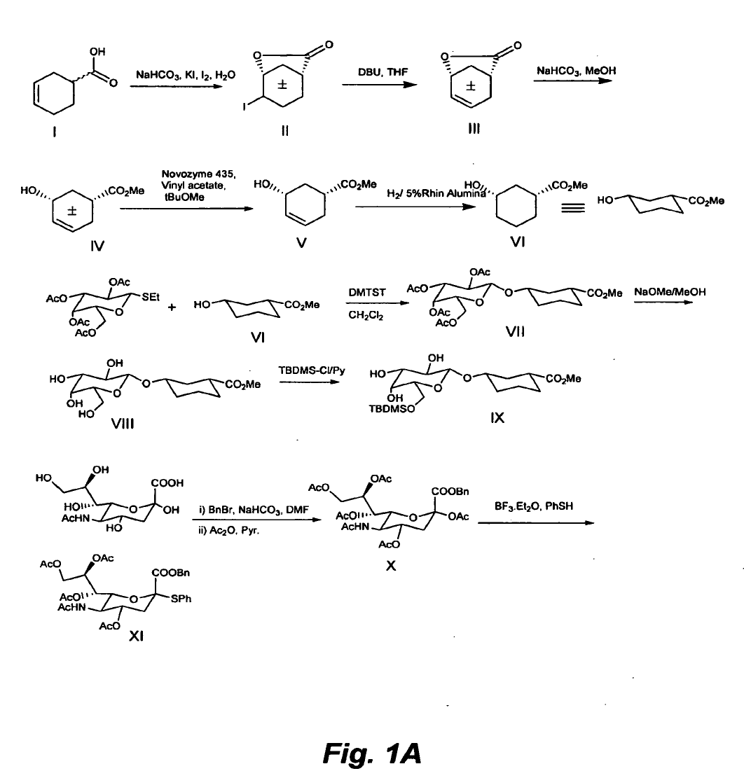

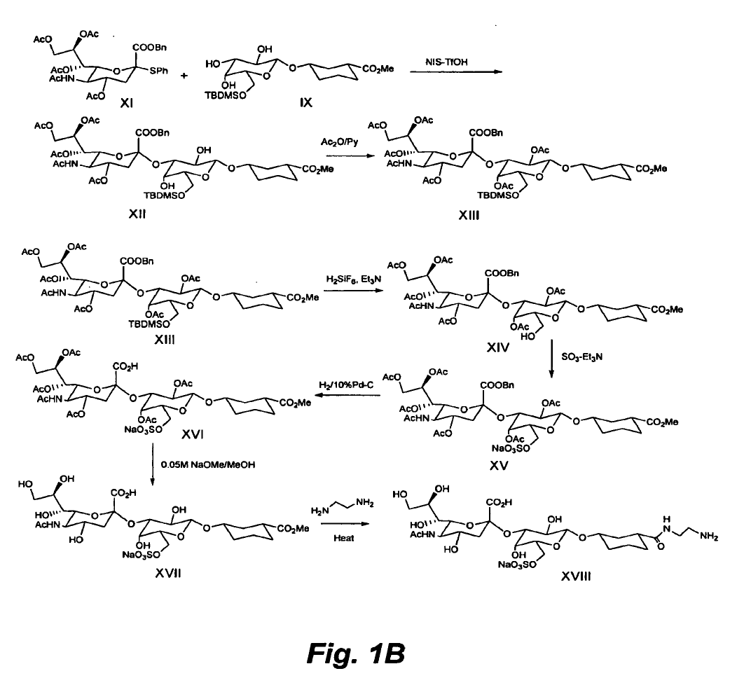

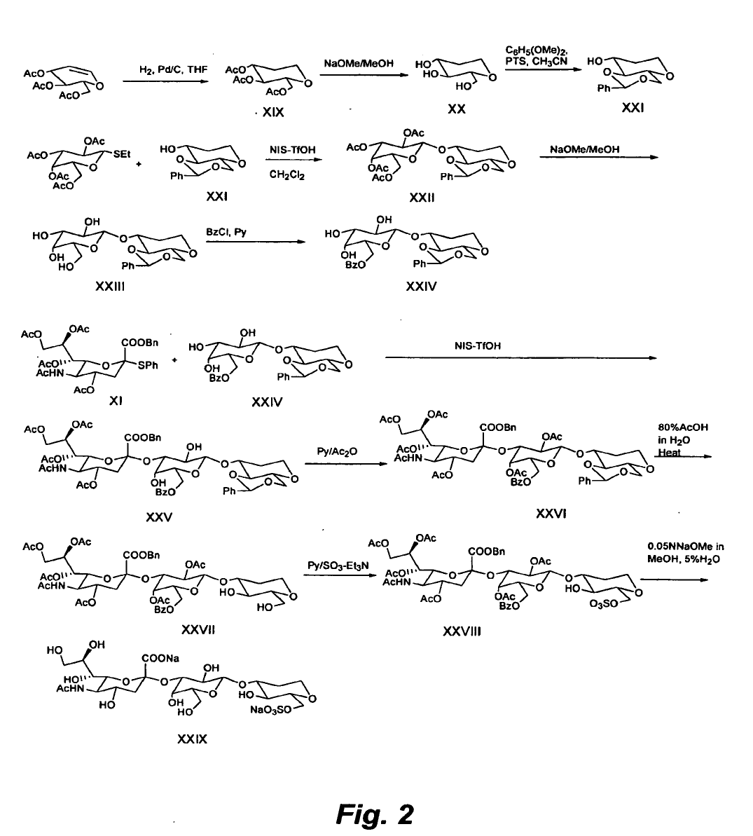

### DETAILED DESCRIPTION

As noted above, the present invention provides Siglec-8 modulating compounds, compositions thereof and methods for modulating or detecting Siglec-8-mediated functions. The compounds disclosed herein do not possess fucose. This is surprising since fucose is believed in the art to be necessary for binding Siglec-8. The compounds disclosed herein may be used in vitro or in vivo to modulate (e.g., inhibit or enhance) Siglec-8-mediated functions in a variety of contexts, discussed in further detail below. Examples of Siglec-8-mediated functions include inflammatory diseases. Inflammatory diseases include asthma, psoriasis and allergy.

Compounds useful in the compositions and methods of the present invention include embodiments with the formula:

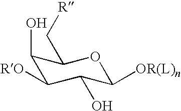

In the above formula, several positions have symbols (rather than chemical elements) indicated in order to permit variability in the substituents at those positions. R may be N-acetylglucoseamine. Alternatively, R may be the following ringed substituent:

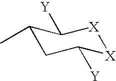

The line (to which no symbol is attached) extending from the ring of this chemical formula represents the point of attachment to the formula above (and does not represent a methyl group). In other words, the line is the chemical bond between the oxygen and R in OR, where R is this ringed substituent. Such a ringed substituent as R possesses two Xs. In an embodiment, one X is oxygen (O) and the other X is CHY where Y is not a ring atom but is attached to C which is a ring atom. In another embodiment, both Xs are CHY. The Y of these embodiments are independently selected from H, OH, NHZ, (CH₂)_{q}OSO₃ where q is generally about 0-4 (including any whole integer range therein), and CH₂Z. Z that is attached to N or C (in NHZ and CH₂Z, respectively) is selected from (or where there are more than one Z then independently selected from) H, acetyl, aliphatic group and aromatic group. As used herein, “independently selected” refers to the selection of identical or different substituents.

“Aliphatic group” refers to straight- or branched-chain hydrocarbons having from 1 to 20 carbon atoms in the chain. Typically there will be 1 to 8 carbon atoms in the chain (C₁-C₈). Examples include methyl, ethyl, propyl, isopropyl, butyl and t-butyl. The aliphatic group (e.g., alkanyl) may be substituted or unsubstituted on the chain, and may include one or more carbon-carbon double bonds (alkenyl) or triple bonds (alkynyl). The aliphatic group may be a cycloaliphatic group.

“Cycloaliphatic group” refers to a cyclic aliphatic group that contains between 3 and 8 carbon atoms and has a single ring or fused rings. The cyclic ring may be substituted or unsubstituted, and may be preceded by one or more CH₂ groups. The cycloaliphatic group may be a heterocyclic aliphatic group. Examples of cycloaliphatic groups include cyclohexyl, piperidinyl, piperazinyl and morpholinyl.

“Heterocyclic aliphatic group” refers to a monocyclic ring or fused rings (i.e., rings which share an adjacent pair of atoms) group having in the ring(s) one or more heteroatoms, preferably selected from nitrogen, oxygen and sulfur. The ring(s) may also have one or more double bonds. However, the ring(s) are not aromatic. The ring(s) may be substituted or unsubstituted, and may be preceded by one or more CH₂ groups.

“Aromatic group” refers to an unsaturated aromatic carbocyclic group of 6 to 14 carbon atoms having a single ring or multiple condensed rings. The aromatic group may be substituted or unsubstituted, and may be preceded by one or more CH₂ groups. The aromatic group may be a heteroaromatic group. Examples of aromatic groups include phenyl, naphthyl, pyridinyl, pyrimidinyl, triazolo, furanyl, oxazolyl, thiophenyl, quinolinyl and diphenyl.

“Heteroaromatic group” refers to a monocyclic or fused ring aromatic group having in the ring(s) one or more heteroatoms, preferably selected from nitrogen, oxygen and sulfur. The heteroaromatic group may be substituted or unsubstituted, and may be preceded by one or more CH₂ groups.

“Alkoxy group” refers to an oxygen substituent possessing an aliphatic group. This is —O-aliphatic; for example methoxy, ethoxy, n-propoxy, i-propoxy, and n-butoxy; and alkenyl or alkynyl variations thereof (except for methoxy). It further refers to the group O-aliphatic-W-aliphatic where W is O or N; for example —O—(CH₂)ₙ—W—(CH₂)ₘ where n and m are independently 1-10.

“Aryloxy group” refers to an oxygen substituent possessing an aromatic group. This is —O-aromatic; for example —O-phenyl, —O-naphthyl, —O-pyridinyl and —O-furanyl.

As noted, any of the above groups may be substituted (i.e., one or more hydrogens is replaced with a substituent). Where there is more than one substituent, they are independently selected. Substituents include a halide (I, Cl, Br or F), an aliphatic group, an aromatic group, an alkoxy group and an aryloxy group.

L is a linker group. There are n linker groups present. Where n is 0, there are no linker groups present in the compound. Where n is 1, there is one linker group present. Where a molecule is attached to the compound through L, n cannot be 0. The linker group may be used to attach a variety of molecules including the compound to form a dimer (homodimer if identical or heterodimer if not), a molecule to make the compound multivalent, a diagnostic agent, and a therapeutic agent. The molecule being attached via L normally possesses a group that is chemically reactive with L, or possesses its own linker group that is reactive with L. For example, where L is an electrophilic group (such as an ester or acid halide), the group chemically reactive with L will be a nucleophilic group, such as —NH₂, —(CH₂)ₚ—NH₂ or —C(═O)—NH—(CH₂)ₚ—NH₂. Alternatively, for example, L may be a nucleophilic group and the group chemically reactive with L will be an electrophilic group. A linker may include a spacer group, such as —(CH₂)ₚ— or —O(CH₂)ₚ— where p is generally about 1-20 (including any whole integer range therein). Other examples of spacer groups include a carbonyl or carbonyl containing group such as an amide.

Embodiments of linkers include the following:

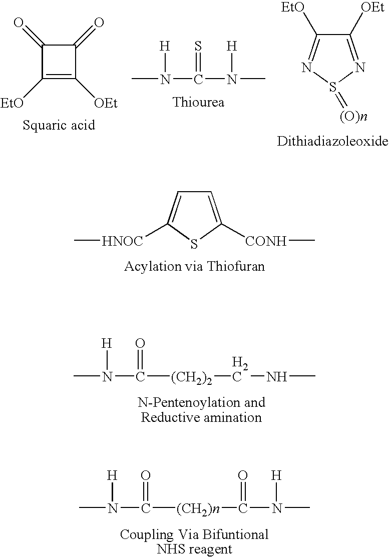

Other linkers, e.g., polyethylene glycols (PEG) or —C(═O)—NH—(CH₂)ₚ—C(═O)—NH₂ where p is as defined above, will be familiar to those in the art or in possession of the present disclosure.

R′ may be H (i.e., taken with the O to form a hydroxyl), sialic acid, or a sialic acid analog. Examples of analogs include:

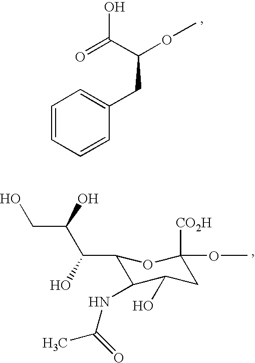

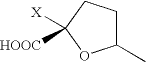

where x is aromatic group,

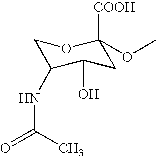

and

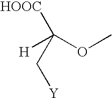

where Y is cyclohexane, t-butane or adamantane.
In the chemical formulae herein, a line extending from an atom depicted or from a carbon implied by the intersection of two other lines, represents the point of attachment (and does not represent a methyl group).

R″ may be OH, sulfated-(A)ₘ group, carboxylated-(A)ₘ group, or phosphorylated-(A)ₘ group. A may be an aliphatic group or an aromatic group. Where m is 0, there is no A and thus a sulfated, carboxylated or phosphorylated group is attached directly to the carbon to which R″ is attached. Where m is 1, A is present and thus a sulfated, carboxylated or phosphorylated group is attached to an aliphatic or aromatic group which in turn is attached to the carbon to which R″ is attached. Examples of sulfated aromatic groups which may be used in the present invention are described in the published U.S. application with Publication No. US-2004-0254145-A1. By replacing the sulfated group(s) in such compounds, for example, with carboxyl or phosphoryl groups, the corresponding carboxylated or phosphorylated aromatic groups, respectively, may be provided.

Siglec-8 modulating compounds as described herein may be present within a pharmaceutical composition. A pharmaceutical composition comprises one or more modulators in combination with (i.e., not covalently bonded to) one or more pharmaceutically or physiologically acceptable carriers, diluents or excipients. Such compositions may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Within yet other embodiments, compositions of the present invention may be formulated as a lyophilizate. Compositions of the present invention may be formulated for any appropriate manner of administration, including for example, topical, oral, nasal, intravenous, intracranial, intraperitoneal, subcutaneous, or intramuscular administration.

A pharmaceutical composition may also, or alternatively, contain one or more active agents, such as drugs (e.g., those set forth herein), which may be linked to (i.e., covalently bonded to) a modulator or may be free within the composition.

The compositions described herein may be administered as part of a sustained release formulation (i.e., a formulation such as a capsule or sponge that effects a slow release of modulating agent following administration). Such formulations may generally be prepared using well known technology and administered by, for example, oral, rectal or subcutaneous implantation, or by implantation at the desired target site. Carriers for use within such formulations are biocompatible, and may also be biodegradable; preferably the formulation provides a relatively constant level of modulating agent release. The amount of modulating agent contained within a sustained release formulation depends upon the site of implantation, the rate and expected duration of release and the nature of the condition to be treated or prevented.

The above described compounds including equivalents thereof may be used in variety of methods of the present invention as it relates to Siglec-8. In one embodiment, the activity of Siglec-8 may be modulated by a method comprising contacting a cell with an effective amount of an above-described compound. The cell may be contacted in vitro or in vivo with the compound.

In another embodiment, a patient who is in need of having inhibited the development of a condition associated with Siglec-8 may be treated by a method comprising administering to the patient an above-described compound in an amount effective to inhibit the development of such a condition. For this method or the above method, the compound may be in combination with a pharmaceutically acceptable carrier or diluent. In other embodiments of this method and the above method, the compound is multivalent by attachment through L, where L is a linker group. Multivalency may be achieved through attachment of additional compound(s), or to a carrier molecule (such as serum albumin or polyethylene glycol) to which a number of the same compound or related compound may be or are already attached. In other embodiments of this method and the above method, the compound is linked by L to a therapeutic agent. An example of a therapeutic agent is a drug. As used herein, the term “drug” refers to any bioactive agent intended for administration to a mammal (human or nonhuman) to prevent or treat a disease or other undesirable condition. Drugs include hormones, growth factors, proteins, peptides and other compounds. Examples of potential drugs include antineoplastic agents (such as 5-fluorouracil and distamycin), integrin agonist/antagonists (such as cyclic-RGD peptide), cytokine agonist/antagonists, histamine agonist/antagonists (such as diphenhydramine and chlorpheniramine), antibiotics (such as aminoglycosides and cephalosporins) and redox active biological agents (such as glutathione and thioredoxin). In other embodiments, therapeutic radionuclides may be linked to a Siglec-8 modulator. In many embodiments, the agent may be linked directly or indirectly to a Siglec-8 modulator.

In another embodiment of the methods of the present invention, an agent may be targeted to a cell expressing Siglec-8 by a method comprising contacting the cell with an above-described compound in an amount effective to target a diagnostic or therapeutic agent to the cell. The agent is linked to the compound by L. The cell may be contacted in vitro or in vivo with the agent linked to the compound.

In another embodiment, a patient who is in need of having an agent targeted to a cell expressing Siglec-8 may be exposed to a method comprising administering to the patient an above-described compound in an amount effective to target a diagnostic or therapeutic agent to the cell. The agent is linked to the compound by L. For this method or the above method, the compound may be in combination with a pharmaceutically acceptable carrier or diluent. As indicated, the agent targeted to the cell may be a diagnostic or therapeutic agent. Therapeutic agents may be a molecule, virus, viral component, cell, cell component or any other substance that can be demonstrated to modify the properties of a target cell so as to provide a benefit for treating or preventing a disorder or regulating the physiology of a patient. A therapeutic agent may also be a prodrug that generates an agent having a biological activity in vivo. Molecules that may be therapeutic agents may be, for example, polypeptides, amino acids, nucleic acids, polynucleotides, steroids, polysaccharides or inorganic compounds. Such molecules may function in any of a variety of ways, including as enzymes, enzyme inhibitors, hormones, receptors, antisense oligonucleotides, catalytic polynucleotides, anti-viral agents, anti-tumor agents, anti-bacterial agents, immunomodulating agents and cytotoxic agents (e.g., radionuclides such as iodine, bromine, lead, palladium or copper). Diagnostic agents include imaging agents such as metals and radioactive agents (e.g., gallium, technetium, indium, strontium, iodine, barium, bromine and phosphorus-containing compounds), contrast agents, dyes (e.g., fluorescent dyes and chromophores) and enzymes that catalyze a colorimetric or fluorometric reaction. In general, therapeutic and diagnostic agents may be attached to a Siglec-8 modulator using a variety of techniques such as those described above. For targeting purposes, a modulator may be administered to a patient as described herein. A modulator may also be used for gene targeting.

Siglec-8 modulators of the present invention may be administered in a manner appropriate to the disease to be treated (including prevented). Appropriate dosages and a suitable duration and frequency of administration may be determined by such factors as the condition of the patient, the type and severity of the patient's disease and the method of administration. In general, an appropriate dosage and treatment regimen provides the modulating agent(s) in an amount sufficient to provide therapeutic and/or prophylactic benefit. Within particularly preferred embodiments of the invention, a Siglec-8 modulator may be administered at a dosage ranging from 0.001 to 1000 mg/kg body weight (more typically 0.01 to 1000 mg/kg), on a regimen of single or multiple daily doses. Appropriate dosages may generally be determined using experimental models and/or clinical trials. In general, the use of the minimum dosage that is sufficient to provide effective therapy is preferred. Patients may generally be monitored for therapeutic effectiveness using assays suitable for the condition being treated (including prevented), which will be familiar to those of ordinary skill in the art.

All compounds of the present invention or useful thereto, include physiologically acceptable salts thereof. Examples of such salts are Na, K, Li, Mg, Ca and Cl.

The following Examples are offered by way of illustration and not by way of limitation.

### EXAMPLES

### Example 1

### Siglec-8 Modulating Compound (XVIII)

Synthesis of Compound II: To a solution of sodium bicarbonate (20 g) in water (325 ml) is added 3-cyclohexene-1-carboxylic acid (10 g) at 0° C. with stirring. To this solution is added an aqueous solution (200 ml) of potassium iodide (79 g) and iodine (21 g) with stirring. After stirring the dark solution for 5 min., chloroform (330 ml) is added and transferred to a separatory funnel. Organic layer is collected and the aqueous layer is extracted (2×300 ml) with chloroform. Combined organic layer is washed with saturated solution of sodium thiosulfate (2×200 ml), dried (anhydrous sodium sulfate), filtered, and concentrated to dryness to give compound II (12 g).

Synthesis of compound III: To a solution of compound II (12 g) in THF (300 ml) is added DBU (11 ml) and the solution is heated at 54° C. overnight. Reaction mixture is poured into 0.5M HCl and extracted with EtOAc (2×300 ml). Organic extracts are dried (anhydrous sodium sulfate), filtered, and concentrated to dryness to give compound III (6 g).

Synthesis of compound IV: To a solution of compound III (6 g) in anhydrous MeOH (80 ml) is added sodium bicarbonate (5 g) and the suspension is stirred for 2 h under reflux. The suspension is filtered and evaporated to dryness. Solid residue is partitioned between water and EtOAc. Aqueous layer is washed with EtOAc (2×) and the combined organic layer is washed sequentially with 1M HCl, brine, and water, dried (anhydrous sodium sulfate), filtered, and concentrated to dryness. Residue is purified by column chromatography (silica gel) to give compound IV (4.5 g).

Synthesis of compound V: A solution of compound IV (4.3 g) in ter-Bu-methyl ether (40 ml) is treated with vinyl acetate (20 g) and Novozyme 435 (256 mg, lipase acrylate resin from Candida Antarctica). The reaction mixture is stirred for 20 h at room temperature, filtered and concentrated to dryness. The residue is purified by column chromatography (silica gel) to afford compound V (2.6 g).

Synthesis of compound VI: To a solution of compound V (2 g) in THF (25 ml) is added 5% Rh in Alumina (500 mg) and reaction mixture is shaken under hydrogen (20 psi) for 24 h. Catalyst is filtered through a celite bed and the filtrate is concentrated to dryness. The residue is purified by column chromatography (silica gel) to give compound VI (1.8 g).

Synthesis of Compound VII: To solution of compound VI (1 g) and commercially available ethyl 1-thio-2,3,4,6-tetra-O-acetyl-β-D-galactopyranoside (3.2) in dichloroethane (25 ml) is added molecular sieves (4 Å, 1 g) and the suspension is stirred at room temperature for 2 h. To this solution is added a solution of DMTST (1 g) in dichloromethane (15 ml) and the reaction mixture is stirred for 40 h at room temperature. It is then filtered through a celite bed and the filtrate is transferred to a separatory funnel, washed with a saturated solution of sodium bicarbonate and water, dried (anhydrous sodium sulfate), filtered, and concentrated to dryness. The residue is purified by column chromatography (silica gel) to give compound VII.

Synthesis of compound VIII: Compound VII (2 g) is treated with 0.025N NaOMe in anhydrous MeOH (20 ml) for 3 h and then neutralized with IR-120 (H⁺) resin. Solvent is evaporated off and concentrated to dryness to give compound VIII.

Synthesis of compound IX: To a solution of compound VIII (1 g) in pyridine (15 ml) is added TBDMS (0.9 g) and the solution is stirred at room temperature for 16 h. Solvent is evaporated off and the residue is purified by column chromatography (silica gel) to give compound IX.

Synthesis of compound X: To a solution of N-acetyl-neuraminic acid (40 g) in DMF (450 ml) is added sodium bicarbonate (20 g) and benzyl bromide (18.5 ml) and the suspension is stirred for 2 h at 60° C. Solvent is evaporated off and coevaporated with toluene (2×300 ml). To the solid residue is added pyridine (300 ml) and acetic anhydride (250 ml). The reaction mixture is stirred at room temperature for 10 h. The solvent is evaporated off and the residue is dissolved in dichloromethane. Solution is washed with 1M HCl, saturated solution of sodium bicarbonate and water, organic layer is dried (anhydrous sodium sulfate), filtered, and concentrated to dryness. The syrupy residue is used for the next step without further purification.

Synthesis of compound XI: Crude compound X is dissolved in dichloromethane (500 ml) and treated with thiophenol (15 ml) in presence of BF₃.Et₂O (20 ml) for 2 h at room temperature. The reaction mixture is washed with saturated solution of sodium bicarbonate and water, dried (anhydrous sodium sulfate), filtered, and concentrated to dryness. The residue is purified by column chromatography (silica gel) to give compound XI (20 g).

Synthesis of compound XII: A solution of compound IX (1 g) and compound XI (1.8 g) in dichloromethane (25 ml) is stirred with powdered molecular sieves (1 g, 4 Å) for 1 h at room temperature. N-iodosuccinimide (1.2 g) is added and the reaction mixture is stirred for another 20 min. at room temperature. The reaction mixture is cooled down to −10° C. A 0.1M solution of trifluoromethanesulfonic acid in dichloromethane (5 ml) is added dropwise to the reaction mixture with stirring. Stirring is continued for 1 h and then filtered through a celite bed. The filtrate is washed successively with saturated solution of sodium thiosulfate, sodium bicarbonate, and brine, dried (anhydrous sodium sulfate), filtered, and concentrated to dryness. The residue is purified by column chromatography (silica gel) to give compound XII.

Synthesis of compound XIII: Compound XII (1 g) is treated with pyridine (10 ml) and acetic anhydride (1 ml) for 24 h at room temperature. Solvent is evaporated off and the residue is purified by column chromatography (silica gel) to give compound XIII.

Synthesis of compound XIV: To a solution of compound XIII (0.8 g) in acetonitrile (10 ml) is added triethylamine (0.05 ml) and H₂SiF₆ (0.3 ml) and the mixture is stirred at room temperature for 2 h. The reaction mixture is diluted with dichloromethane (50 ml) and the organic layer is washed with a saturated solution of sodium bicarbonate and water, dried (anhydrous sodium sulfate), filtered, and concentrated to dryness. The residue is purified by column chromatography (silica gel) to give compound XIV.

Synthesis of compound XV: Compound XIV (0.5 g) is dissolved in pyridine (5 ml) and a solution of sulfurtrioxide-triethylamine complex (0.5 g) in pyridine is added at room temperature for 10 h. The reaction mixture is diluted with dichloromethane (50 ml). The organic layer is washed with a saturated solution of sodium bicarbonate, dried (anhydrous sodium sulfate), filtered, and concentrated to dryness. The residue is purified by column chromatography (silica gel) to give compound XV.

Synthesis of compound XVI: Compound XV (0.5 g) is dissolved in MeOH (10 ml) and added 10% Pd—C (0.5 g), the suspension is shaken under hydrogen (55 psi) for 48 h. The reaction mixture is filtered off and the filtrate is concentrated to dryness to give compound XVI.

Synthesis of compound XVII: Compound XVI (0.3 g) is treated with 0.025M NaOMe in MeOH (5 ml) for 2 h at room temperature, neutralized with IR-120 (H⁺) resin, filtered, and concentrated to dryness. The residue is purified by sephadex G-10 to give compound XVII.

Synthesis compound XVIII: Compound XVII (0.1 g) is dissolved in ethylenediamine (5 ml) and heated for 2 h at 65° C. Solvent is evaporated off and the residue is passed through a sephadex G-10 column to give compound XVIII.

### Example 2

### Siglec-8 Modulating Compound (XXIX)

Synthesis of compound XXII: Starting from commercially available 3,4,6-tri-O-acetyl-glucal (20 g), compound XXII (16 g) is synthesized via intermediates XIX, XX, and XXI by exactly the same procedure as described in the literature (J. Med. Chem., 1999, 42, 4909-4913)

Synthesis of compound XXIII: To a solution of compound XXII (15 g) is added 0.025M NaOMe in MeOH (150 ml) and the solution is stirred for 4 h at room temperature. It is then neutralized with IR-120(H⁺) resin, filtered, and concentrated to dryness to afford compound XXIII (9 g).

Synthesis of compound XXIV: A solution of compound XXIII (2 g) in pyridine (20 ml) is cooled down to −20° C. To this solution is added a solution of benzoyl chloride (0.8 g) in pyridine (8 ml) dropwise during 2 h with stirring at −20° C. Temperature is gradually increased to room temperature during 3 h and allowed to stir at room temperature for another 2 h. MeOH (0.5 ml) is added and the reaction mixture is concentrated to dryness. The residue is purified by column chromatography (silica gel) to give compound XXIV.

Synthesis of compound XXV: Compound XXIV (1 g) is reacted with compound XI (2 g) exactly in same way as described for the synthesis of compound XII to afford compound XXV after purification by column chromatography (silica gel).

Synthesis of compound XXVI: Compound XXV (0.5 g) is treated with pyridine (2 ml) and acetic anhydride (1.5 ml) for 20 h. Solvent is evaporated off to give a syrupy residue. This is used for the next step without further purification.

Synthesis of compound XXVII: Compound XXVI (0.5 g) is treated with 80% acetic in water for 2 h at 80° C. Solvent is evaporated off and the residue is purified by column chromatography (silica gel) to give compound XXVII.

Synthesis of compound XXVIII: Compound XVII (0.25 g) is dissolved in pyridine (2.5 ml) and a solution of sulfur trioxide-triethylamine complex (0.25 g) in pyridine is added at room temperature for 10 h. The reaction mixture is diluted with dichloromethane (25 ml). The organic layer is washed with a saturated solution of sodium bicarbonate, dried (anhydrous sodium sulfate), filtered, and concentrated to dryness. The residue is purified by column chromatography (silica gel) to give compound XXVIII.

Synthesis of compound XXIX: Compound XXVIII (0.15 g) is treated with 0.01N NaOMe in MeOH (2 ml) for 1 h. After 1 h, 0.1 ml water is added and the reaction mixture is stirred for additional 1 h at room temperature. The reaction mixture is neutralized with IR-120 (H⁺) resin, filtered, and concentrated to dryness. The residue is purified by passing through a sephadex G-10 column to give compound XXIX.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention.

1. A method for modulating the activity of Siglec-8 comprising contacting a cell with an effective amount of a compound with the formula: 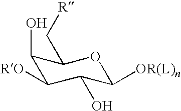
wherein
R═N-acetylglucoseamine or 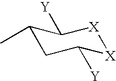
wherein both X are CHY, or one X is O and the other X is CHY, where the Y are independently selected from H, OH, NHZ, (CH₂)_{q}OSO₃, and CH₂Z, where Z is selected from or are independently selected from H, acetyl, aliphatic group and aromatic group;
q=0-4;
L=linker group;
n=0-1;
R′═H, sialic acid, or a sialic acid analog;
R″═OH, sulfated-(A)ₘ group, carboxylated-(A)ₘ group, or phosphorylated-(A)ₘ group;
A=aliphatic or aromatic group; and
m=0-1.

2. A method of treating a patient who is in need of having inhibited the development of a condition associated with Siglec-8, comprising administering to the patient a compound in an amount effective to inhibit the development of such a condition, the compound with the formula: 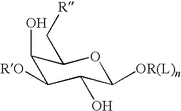
wherein
R═N-acetylglucoseamine or 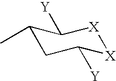
wherein both X are CHY, or one X is O and the other X is CHY, where the Y are independently selected from H, OH, NHZ, (CH₂)_{q}OSO₃, and CH₂Z, where Z is selected from or are independently selected from H, acetyl, aliphatic group and aromatic group;
q=0-4;
L=linker group;
n=0-1;
R′═H, sialic acid, or a sialic acid analog;
R″═OH, sulfated-(A)ₘ group, carboxylated-(A)ₘ group, or phosphorylated-(A)ₘ group;
A=aliphatic or aromatic group; and
m=0-1.

3. The method of claim 1 or 2 wherein the compound is in combination with a pharmaceutically acceptable carrier or diluent.

4. The method of claim 1 or 2 wherein the compound is multivalent by attachment through L.

5. The method of claim 4 wherein the compound is in combination with a pharmaceutically acceptable carrier or diluent.

6. The method of claim 1 or 2 wherein the compound is linked by L to a therapeutic agent.

7. The method of claim 6 wherein the compound is in combination with a pharmaceutically acceptable carrier or diluent.

8. A method of targeting an agent to a cell expressing Siglec-8, comprising contacting the cell with a compound in an amount effective to target a diagnostic or therapeutic agent to the cell, wherein the agent is linked by L to the compound with the formula: 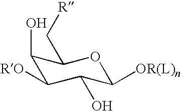
wherein
R═N-acetylglucoseamine or 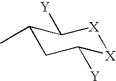
wherein both X are CHY, or one X is O and the other X is CHY, where the Y are independently selected from H, OH, NHZ, (CH₂)_{q}OSO₃, and CH₂Z, where Z is selected from or are independently selected from H, acetyl, aliphatic group and aromatic group;
q=0-4;
L=linker group;
n=1;
R′═H, sialic acid, or a sialic acid analog;
R″═OH, sulfated-(A)ₘ group, carboxylated-(A)ₘ group, or phosphorylated-(A)ₘ group;
A=aliphatic or aromatic group; and
m=0-1.

9. The method of claim 8 wherein the agent is a diagnostic agent.

10. The method of claim 8 wherein the agent is a therapeutic agent.

11. A method of targeting an agent to a cell expressing Siglec-8 in a patient in need thereof, comprising administering to the patient a compound in an amount effective to target a diagnostic or therapeutic agent to the cell, wherein the agent is linked by L to the compound with the formula: 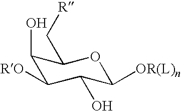
wherein
R═N-acetylglucoseamine or 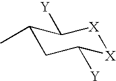
wherein both X are CHY, or one X is O and the other X is CHY, where the Y are independently selected from H, OH, NHZ, (CH₂)_{q}OSO₃, and CH₂Z, where Z is selected from or are independently selected from H, acetyl, aliphatic group and aromatic group;
q=0-4;
L=linker group;
n=1;
R′═H, sialic acid, or a sialic acid analog;
R″═OH, sulfated-(A)ₘ group, carboxylated-(A)ₘ group, or phosphorylated-(A)ₘ group;
A=aliphatic or aromatic group; and
m=0-1.

12. The method of claim 11 wherein the agent is a diagnostic agent.

13. The method of claim 11 wherein the agent is a therapeutic agent.

14. The method of any one of claims 8, 9, 10, 11, 12 or 13 wherein the compound is in combination with a pharmaceutically acceptable carrier or diluent.

15.-16. (canceled)
